(19) United States
(12) Patent Application Publication
(10) Pub. No.: US 2024/0390074 A1
(43) Pub. Date: Nov. 28, 2024

(54) SYSTEMS AND METHODS FOR DETERMINING ONE OR MORE PHASES OF ANATOMICAL MOVEMENT

(71) Applicant: Medtronic Navigation, Inc., Lafayette, CO (US)

(72) Inventors: Anthony B. Ross, Boulder, CO (US); Mark L. Stiger, Santa Rosa, CA (US); Osvaldo Andres Barrera, Madison, CT (US); Bradley W. Jacobsen, Erie, CO (US); Yvan R. Paitel, Louisville, CO (US); Patrick A. Helm, Canton, MA (US); Kaustubh R. Patil, Plymouth, MN (US); Joshua J. Blauer, White Bear Township, MN (US); Trevor C. Huang, Maple Grove, MN (US); Joseph D. Brannan, Lyons, CO (US); Elliot C. Schmidt, Minneapolis, MN (US)

(21) Appl. No.: 18/656,752

(22) Filed: May 7, 2024

Related U.S. Application Data
(60) Provisional application No. 63/468,976, filed on May 25, 2023.

(51) Int. Cl. A61B 34/20; A61B 5/11; A61B 5/113

(52) U.S. Cl. CPC A61B 34/20; A61B 5/11; A61B 5/113; A61B 2034/2048; A61B 2034/2051

(57) ABSTRACT

Systems and methods for determining one or more phases of anatomical movement are provided. A catheter positioned in an anatomical element may be tracked using a tracker configured to track a pose of the catheter. Sensor data corresponding to a plurality of pose information of the catheter may be received from the tracker. One or more phases of motion of the anatomical element may be determined based on the plurality of pose information. An image may be received at a target phase of the one or more phases.

### CROSS-REFERENCE TO RELATED APPLICATION

This application claims the benefit of and priority to U.S. Provisional Patent Application No. 63/468,976 filed May 25, 2023, the entire disclosure of which is incorporated by reference herein.

### BACKGROUND

The present disclosure is generally directed to tracking movement of an anatomical element, and relates more particularly to tracking movement of an anatomical element to determine one or more phases of anatomical movement.

Surgical robots may assist a surgeon or other medical provider in carrying out a surgical procedure, or may complete one or more surgical procedures autonomously. Imaging may be used by a medical provider for diagnostic and/or therapeutic purposes. Patient anatomy can change over time, particularly following placement of a medical implant in the patient anatomy.

### BRIEF SUMMARY

Example aspects of the present disclosure include:

A system for determining one or more phases of anatomical movement according to at least one embodiment of the present disclosure comprises a catheter positioned in an anatomical element; a tracker for tracking a pose of the catheter; a processor; and a memory storing data for processing by the processor, the data, when processed, causing the processor to: track the catheter; receive sensor data corresponding to a plurality of pose information of the catheter from the tracker; determine one or more phases of motion of the anatomical element based on the plurality of pose information; receive an image at a target phase of the one or more phases; and display the image.

Any of the aspects herein, wherein tracking the catheter comprises tracking a speed of the catheter.

Any of the aspects herein, wherein at least one of the tracker, the catheter, or a combination of the tracker and the catheter is shaped to at least one of augment, decrease, or increase motion caused by fluid flow in the anatomical element.

Any of the aspects herein, wherein the memory stores further data for processing by the processor that, when processed, causes the processor to: receive bio-electric signals, wherein determining the one or more phases of motion is further based on the bio-electric signals.

Any of the aspects herein, wherein the memory stores further data for processing by the processor that, when processed, causes the processor to: receive respiratory cycle signals, wherein determining the one or more phases of motion is further based on the bio-electric signals.

Any of the aspects herein, wherein the anatomical element comprises a heart and the one or more phases correspond to one or more phases of the heartbeat.

Any of the aspects herein, wherein the one or more phases of the heartbeat comprise at least a diastole motion and a systole motion.

Any of the aspects herein, wherein the memory stores further data for processing by the processor that, when processed, causes the processor to: average the sensor data for a target phase of the one or more phases; and apply the average to subsequent tracking at the target phase.

Any of the aspects herein, wherein the memory stores further data for processing by the processor that, when processed, causes the processor to determine a patient state based on the motion of the anatomical element.

Any of the aspects herein, wherein the memory stores further data for processing by the processor that, when processed, causes the processor to: determine movement and a velocity of the catheter; and determine an environmental state of the catheter based on the movement and velocity.

Any of the aspects herein, wherein the tracker comprises at least one of an inertial measurement unit tracker, an electromagnetic tracker, or a combination of the inertial measurement unit tracker and the electromagnetic tracker.

Any of the aspects herein, wherein the tracker comprises an imaging device and the sensor data comprises image data, wherein the plurality of pose information is derived from the image data.

A system for determining one or more phases of anatomical movement according to at least one embodiment of the present disclosure comprises a catheter positioned in an anatomical element; a tracker for tracking a pose of the catheter; a processor; and a memory storing data for processing by the processor, the data, when processed, causing the processor to: track the catheter; receive sensor data corresponding to a plurality of pose information of the catheter from the tracker; and determine one or more phases of motion of the anatomical element based on the plurality of pose information.

Any of the aspects herein, wherein the memory stores further data for processing by the processor that, when processed, causes the processor to receive an image at a phase of the one or more phases and display the image

Any of the aspects herein, wherein the tracker comprises at least one of an inertial measurement unit tracker or an electromagnetic tracker.

Any of the aspects herein, wherein the memory stores further data for processing by the processor that, when processed, causes the processor to determine a patient state based on the motion of the anatomical element.

Any of the aspects herein, wherein the memory stores further data for processing by the processor that, when processed, causes the processor to: determine movement and a velocity of the catheter; and determine an environmental state of the catheter based on the movement and velocity.

A system for correlating one or more images with one or more cycles of motion of an anatomical element according to at least one embodiment of the present disclosure comprises a processor; and a memory storing data for processing by the processor, the data, when processed, causing the processor to: receive a plurality of images over a period of time; receive sensor data corresponding to a plurality of pose information pose of a catheter corresponding to pose information of the catheter over the period of time to yield; determine one or more first motion phases of a first motion of the anatomical element based on the plurality of pose information; determine one or more second motion phases of a second motion of the anatomical element based on the plurality of pose information; and determine a subset of images from the plurality of images corresponding to a target first motion phase of the one or more first motion phases of the first motion and a target second motion phase of the one or more second motion phases of the second motion.

Any of the aspects herein, wherein the subset of images comprises a first subset of images, and wherein the memory stores further data for processing by the processor that, when processed, causes the processor to determine a second subset of images from the plurality of images corresponding to another target first motion phase the one or more first motion phases of the first motion and another target second motion phase of the one or more second motion phases of the second motion.

Any of the aspects herein, wherein the memory stores further data for processing by the processor that, when processed, causes the processor to reconstruct a three-dimensional model based on an intersection of the first subset of images and the second subset of images.

Any of the aspects herein, wherein the memory stores further data for processing by the processor that, when processed, causes the processor to display at least one of the first subset of images, the second subset of images, or a combination of the first subset of images and the second subset of images.

Any of the aspects herein, wherein the first motion corresponds to a cardiac cycle and the second motion corresponding to a respiratory cycle.

Any of the aspects herein, wherein the memory stores further data for processing by the processor that, when processed, causes the processor to receive respiratory information of a patient over the period of time, wherein determining the one or more second motion phases of a second motion is also based on the respiratory information.

Any of the aspects herein, wherein the memory stores further data for processing by the processor that, when processed, causes the processor to obtain and display a subsequent plurality of images over a subsequent period of time at the target first motion phase and the target second motion phase.

Any of the aspects herein, wherein the memory stores further data for processing by the processor that, when processed, causes the processor to: average sensor data at a target phase of the one or more phases; and apply the average to subsequent tracking at the target phase.

Any of the aspects herein, wherein the sensor data comprises at least one of image data, accelerometer data, pose data, or motion data.

Any of the aspects herein, wherein the memory stores further data for processing by the processor that, when processed, causes the processor to: receive bio-electric signals, wherein determining the one or more phases of motion is further based on the bio-electric signals.

Any of the aspects herein, wherein the memory stores further data for processing by the processor that, when processed, causes the processor to: receive respiratory cycle signals, wherein determining the one or more phases of motion is further based on the bio-electric signals.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is a schematic diagram of a system according to at least one embodiment of the present disclosure;

FIG. 3 is a flowchart according to at least one embodiment of the present disclosure;

FIG. 4 is a flowchart according to at least one embodiment of the present disclosure;

FIG. 5 is a flowchart according to at least one embodiment of the present disclosure; and

FIG. 6 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Placement of implantable devices such as cardiac devices using catheters (or any other device or component for placement) requires accurate location of the catheter inside a patient's anatomy. O-arm or computed tomography (CT) imaging can be used to help confirm placement of the implantable device (e.g., cardiac device) and the catheter, however, the O-arm or CT imaging devices may be cumbersome and also exposes the patient and surgical staff to radiation. A physician placing the catheter would benefit from quick and easy to use methods to gather information about a position and/or orientation of the catheter without exposing the patient and the surgical staff to radiation.

At least one embodiment of the present disclosure provides for systems and methods for utilizing spatial temporal information from tracked catheters using trackers such as, for example, electromagnetic (EM) trackers or inertial measurement unit (IMU) trackers to provide clinical information about the location of the catheter. EM and/or IMU sensors are placed on, for example, cardiac catheters to provide a constant stream of positional and orientational information on the heart. More specifically, the location of the catheter changes (as the catheter moves) due to the beating of the heart in a cyclic manner and this motion can be recorded and graphed. The motion can then be mapped to the cardiac cycle similar to an electrocardiogram (EKG or ECG) that can be mapped to the cardiac cycle. Further, the velocity of the device can be derived from the trackers. Thus, knowing the velocity and location of the device can be used to improve placement of implantable devices such as cardiac devices.

The systems and methods using position and orientation (e.g., pose information) and/or velocity information from the tracked catheters can also be used to improve EM and/or IMU tracking accuracy by timing the acquisition of the EM and/or IMU signal to a specific time (e.g., phase) in the cardiac signal. For example, the EM tracker may be most accurate during diastole and may have too much motion during systole to accurately navigate the EM tracker. Timing the EM and/or IMU data acquisition to match cycle levels of less motion can improve an accuracy of the tracking. In another example, as the EM tracker measures the heartbeat cycles, a tracking or navigation system may average, correlate, or otherwise combine EM and/or IMU samples to accurately navigate the tracker throughout the entire cycle including during systole motion.

The systems and methods can also be used to replace or improve information retrieved from the EKG by showing systole and diastole movement of the heart measured at specific anatomical locations. The systems provided may be simplified and use less components as the catheter does not need to be connected to the EKG to obtain cardiac information as the cardiac information can be determined using the EM and/or IMU tracking and software to derive the cardiac information from the EM and/or IMU data.

The systems and methods can also be used to time acquisition of image(s) such as ultrasound images or CT images at a target phase of the heartbeat cycle to reduce motion artifact.

The systems and methods can also be used to correlate or identify specific anatomical locations. For example, the catheter will experience more motion in a center of blood flow of the aorta as compared to near a side wall of the aorta. In another example, motion in the ventricle should be different than motion in the atrium. Thus, specific regions of the heart wall may have distinct motion patterns that can help confirm that a tip of the catheter is in a target location.

The systems and methods can also be used to identify or correlate specific disease states. For example, low motion or velocity may indicate low blood flow rates.

The systems and methods can also be used to ensure that motion of the implantable device or cardiac device does not exceed known limits of the device. For example, the EM and/or IMU data can be used to determine if force and/or torque limits are exceeded on the cardiac device (or any implantable device) and if so, then a user can be alerted. Similarly, the environment or motion of the catheter can be checked or confirmed before delivery of the cardiac device such as, for example, a micra device or heart valve. Normal values for motion of the tracked catheter can be confirmed prior to delivery of the cardiac device and if the values or abnormally large or small the user can be alerted.

In addition to the heartbeat cyclic motion, the shape, size, and location of the heart and other organs change dynamically as the heart beats and the lungs breathe. In other words, the cardiac cycle and the respiratory cycle can affect the heart and/or other organs. This movement can affect patient imaging (such as, for example, during ultrasound imaging) as sequential or consecutive images correspond to different stages/phases of the cardiac cycle and the respiratory cycle. This correspondence results in mismatching between sequential images during image modeling (e.g., ultrasound 2D, 3D, 4D, 2D+Doppler) as a position/size/shape of the organs(s) will not match through consecutive images. Registration may also be affected as the position/size/shape of the organ(s) do not match from consecutive images to volumetric images. Additionally, planning and execution of a surgical procedure using image guided surgery may be affected as anatomy previously imaged may not match a current position/size/shape of the organs.

At least one embodiment of the present disclosure provides for systems and methods for dynamically synchronizing acquired images with the cardiac cycle and the respiratory cycle to create multiple groups of non-consecutive images and image models with matching phases (e.g., matching organ location, size, and shape). These systems and methods can be applied to, for example, images such as ultrasound images captured in real-time and from different perspectives/locations relative to the organ(s).

The cardiac cycle and the respiratory cycle can be measured using, for example, current patient monitoring (e.g., respiratory monitoring/controls, cardiac monitoring/controls, ECG, EKG, etc.), patient and/or instrument tracking using EM tracking (e.g., magnetic induction coil, etc.), patient and/or instrument tracking using IMU tracking (e.g., accelerometer, gyroscope, etc.), computing devices to interpret ECG graphs dynamically shown on a screen of some ultrasound imaging devices, or combinations thereof. In some instances, combined respiratory monitoring (e.g., tidal volume) and patient electromagnetic or inertial tracking or both (e.g., thoracic or abdominal movements) may be used to determine movements during the respiratory cycle. Similarly, combined cardiac monitoring (e.g., ECG) and patient electromagnetic or inertial tracking or both (e.g., anterior/posterior lateral movements) may be used to determine movements during the cardiac cycle. In other instances, combined cardiac monitoring (e.g., ECG) and instrument electromagnetic or inertial tracking or both (e.g., tracked catheters within the heart) may be used to determine movements during the cardiac cycle.

The cardiac cycle and the respiratory cycle can be synchronized with imaging such as, for example, ultrasound imaging using direct communication between multiple systems (e.g., navigation system, imaging devices, computing devices, etc.), indirect communication between multiple systems, and/or correlations between multiple systems.

The systems and methods for dynamically synchronizing acquired images with the cardiac cycle and the respiratory cycle can be used to improve imaging, modeling, registration, and image guidance. For example, the imaging, modeling, registration, and image guidance can be improved using averaged images with selected phases, correlated images throughout a cycle, displayed images at a chosen phase (e.g., strobing or freezing ultrasound images, dynamic ultrasound strobe effect to freeze image at a specific point (e.g., target phase) in the cardiac rhythm while allowing dynamic movement of instruments relative to the anatomical structures, hide ultrasound images taken outside of the target phase phase, highlight selected phase ultrasound images and dim out-of-phase ultrasound images, matching the phase of a phased CT or MRI, etc.), reconstructed images and models with selected phases or through a cyclical motion (e.g., reconstruct a 3D heart model at a selected cardiac phase, reconstruct a region of interest cyclical motion using speckle ultrasound tracking, etc.), and/or adjusted regions of interest or targets for cyclical motions (e.g., synchronizing electronic control of ultrasound imaging fan direction or depth or both with cardiac motions, etc.).

The systems and methods for dynamically synchronizing acquired images with the cardiac cycle and the respiratory cycle can also be used to improve EM and/or IMU tracking. The EM and/or IMU tracking can be improved using, for example, averaged samples within the same phases, correlated samples throughout the cardiac cycle and the respiratory cycle, displayed tracking at a chosen target phase (e.g., strobing or freezing tracked positions and orientations, dynamic ultrasound strobe effect to freeze tracking at a specific point in the cardiac rhythm while allowing dynamic movement of structures relative to, for example, instruments, hide tracking taken outside of selected phase, highlight selected phase tracking and dim out-of-phase tracking, etc.), reconstructed tracking with selected phases or through a cyclical motion (e.g., reconstruct a tracked heart wall motion throughout the cardiac cycle, etc.), and/or adjusted regions of interest or targets for cyclical motions.

Embodiments of the present disclosure provide technical solutions to one or more of the problems of (1) determining cardiac and/or respiratory motion, (2) accurately placing an implantable device in a patient, (3) determining an environmental state for the implantable device, and (4) improving displayed images and image reconstruction.

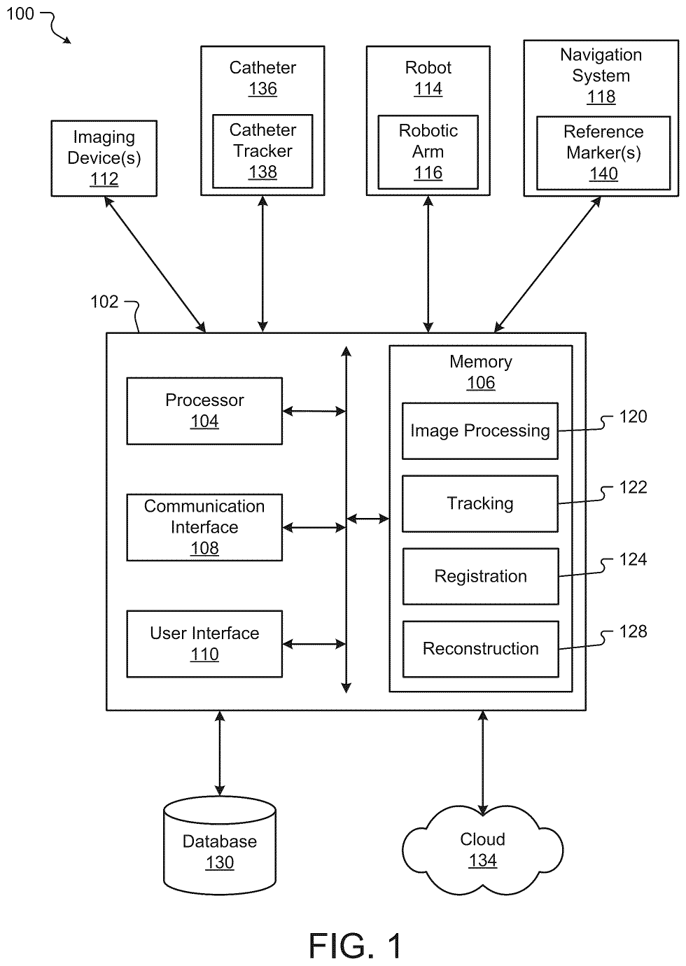

Turning first to FIG. 1, a block diagram of a system 100 according to at least one embodiment of the present disclosure is shown. The system 100 may be used to track movement of an anatomical element and determine one or more phases of motion of the anatomical element, and/or carry out one or more other aspects of one or more of the methods disclosed herein. The system 100 comprises a computing device 102, one or more imaging devices 112, a catheter 136 (trackable with a catheter tracker 138), a robot 114, a navigation system 118, a database 130, and/or a cloud or other network 134. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the catheter tracker 138, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the methods 300, 400, 500 and/or 600 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, tracking 122, registration 124, and/or reconstruction 128.

The image processing 120 enables the processor 104 to process image data of an image (received from, for example, the imaging device 112, an imaging device of the navigation system 118, or any imaging device) for the purpose of, for example, identifying information about anatomical elements and/or objects depicted in the image. The information may comprise, for example, identification of hard tissue and/or soft tissues, a boundary between hard tissue and soft tissue, a boundary of hard tissue and/or soft tissue, identification of a surgical tool or a reference marker 140, etc. The image processing 120 may, for example, identify hard tissue, soft tissue, and/or a boundary of the hard tissue and/or soft tissue by determining a difference in or contrast between colors or grayscales of image pixels. For example, a boundary between the hard tissue and the soft tissue may be identified as a contrast between lighter pixels and darker pixels.

The tracking 122 enables the processor 104 to track the catheter tracker 138 which may comprise, for example, EM and/or IMU trackers. The tracking 122 may, for example, enable the processor 104 to receive sensor data from the catheter tracker 138 and determine pose information (e.g., position and orientation) of the catheter 136 from the sensor data. The tracking 122 may also, for example, enable the processor 104 to determine a velocity of the catheter 136 from the sensor data.

The registration 124 enables the processor 104 to correlate an image with another image. The registration 124 may enable the processor 104 to also correlate identified anatomical elements and/or objects in one image with identified anatomical elements and/or objects in another image. The registration 124 may enable information about the anatomical elements and the objects (e.g., the catheter 136, for example) to be obtained and measured.

The reconstruction 128 enables the processor 104 to generate a three-dimensional (3D) representation of one or more anatomical elements and/or one or more objects based on one or more image(s) received by the processor 104. Generating the 3D representation by the processor 104 may include determining a surface representation or virtual boundary of the one or more anatomical elements and/or the one or more objects depicted in image(s) received by the processor 104 and based on corresponding image pose information. More specifically, in some embodiments, each image may be positioned adjacent to another image based on the respective corresponding image pose information and a surface representation may be formed based the relative position of surfaces depicted in each image. In some embodiments, the surface representation may be a virtual mesh. The virtual mesh may comprise, for example, a set of polygonal faces that, when taken together, form a surface covering of a virtual object. The set of polygonal faces may be connected at their edges and vertices to define a shape of the virtual object.

Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the catheter tracker 138, the robot 114, the database 130, and/or the cloud 134.

The computing device 102 may also comprise a communication interface 108. The communication interface 108 may be used for receiving image data or other information from an external source (such as the imaging device 112, the catheter tracker 138, the robot 114, the navigation system 118, the database 130, the cloud 134, and/or any other system or component not part of the system 100), and/or for transmitting instructions, images, or other information to an external system or device (e.g., another computing device 102, the imaging device 112, the catheter tracker 138, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The catheter 136 may be any catheter 136 that can be inserted and positioned in a patient and more specifically, may be inserted into an anatomical element of the patient. For example, the catheter 136 may be inserted into a patient's heart, though it will be appreciated that the catheter 136 can be positioned in any anatomical element or region of the patient (e.g., intrathecally in the spinal region of the patient, in the brain region of the patient, etc.). In some instances, the catheter 136 can be implantable. In some embodiments, the catheter 136 may enable fluid sampling from the patient and/or delivery of therapeutics to the patient.

The catheter tracker 138 may comprise one or more sensors disposed on the catheter 136 and configured to track a pose and/or movement of the catheter 136. The catheter tracker 138 may comprise, for example, EM and/or IMU sensors. In other embodiments, the catheter tracker 138 may include one or more or any combination of components that are electrical, mechanical, electro-mechanical, magnetic, electromagnetic, or the like. For example, the catheter tracker 138 may include infrared tracking, light diffraction tracking (e.g., optic fiber), and/or impedance tracking. The catheter tracker 138 may be positioned at a tip of the catheter 136, though in other embodiments, the catheter tracker 138 may be positioned on any portion of the catheter 136. The catheter tracker 138 may be shaped to augment, decrease, and/or increase motion caused by fluid flow in the anatomical element. For example, the catheter tracker 138 may include fins and/or baffles to change the motion of the fluid flow.

The catheter tracker 138 may include a plurality of sensors and each sensor may be positioned at the same location or a different location as any other sensor. In some embodiments, the catheter tracker 138 may include a memory for storing sensor data. In still other examples, the catheter tracker 138 may output signals (e.g., sensor data) to one or more sources (e.g., the computing device 102, the navigation system 114, and/or the robot 116). For example, the catheter tracker 138 may send the data to the computing device 102 when the catheter tracker 138 detects movement of the catheter 136. Further, in some embodiments, the catheter tracker 138 may send data to the computing device 102 to display on the user interface 110 or otherwise notify the surgeon or operator of the change in the characteristic. In other embodiments, the catheter tracker 138 may alert the surgeon or operator of the change in the characteristic by an alert such as, but not limited to, a sound or a light display.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces. The robotic arm(s) 116 may comprise one or more sensors that enable the processor 104 (or a processor of the robot 114) to determine a precise pose in space of the robotic arm (as well as any object or element held by or secured to the robotic arm).

In some embodiments, reference markers (e.g., navigation markers) 140 may be placed on the robot 114 (including, e.g., on the robotic arm 116), the imaging device 112, the patient, or any other object in the surgical space. The reference markers 140 may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers 140, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors or trackers or inertial measurement unit trackers (e.g., the catheter tracker 138). In various embodiments, the navigation system 118 may be used to track a position and orientation (e.g., a pose) of the catheter 136 via the catheter tracker(s) 138, the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the methods 300, 400, 500 and/or 600 described herein. The system 100 or similar systems may also be used for other purposes.

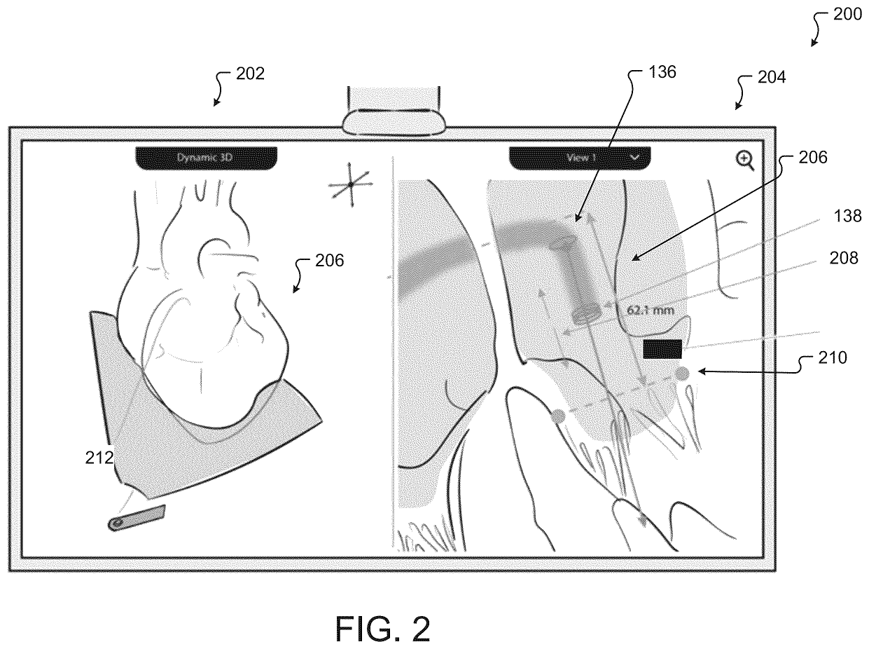

Turning to FIG. 2, a display 200 showing a first screen 202 and a second screen 204 are shown. The display 200 may be the same as or similar to the user interface 110. The first screen 202 displays an image of an anatomical element 212 comprising, for example, a heart 206 of a patient. The second screen 204 displays a detailed view of the heart 206 and a catheter such as the catheter 136 positioned in in the heart 206. As shown, the catheter tracker 138 includes an EM coil wrapped around a tip of the catheter 136. It will be appreciated that in other embodiments the catheter tracker 138 may comprise, for example, an IMU tracker or a combination of an EM tracker and an IMU tracker. It will also be appreciated that in other embodiments the catheter tracker 138 may be positioned anywhere on the catheter 136. As the heart 206 beats, the catheter 136 and the catheter tracker 138 move with the heart 206 and movement (as represented by the arrow 208) of the catheter tracker 138 can be recorded. An annulus displacement (as represented by the arrow 210) is also shown and correlates to a relative displacement of an annulus structure as it moves during a heartbeat. The information that can be obtained from the catheter tracker 136 such as, for example, pose information, movement, a velocity of the catheter tracker 136 can be useful in many cases such as, for example, placement of a device, improvements in tracking and imaging, reconstruction, displaying real-time or near real-time imaging, etc. as will be discussed in detail below.

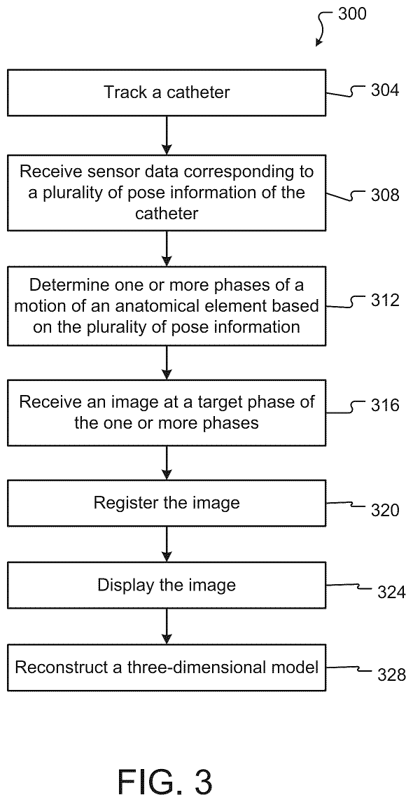

FIG. 3 depicts a method 300 that may be used, for example, for tracking movement of an anatomical element and determining one or more phases of the anatomical movement.

The method 300 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 300. The at least one processor may perform the method 300 by executing elements stored in a memory such as the memory 106. The elements stored in memory and executed by the processor may cause the processor to execute one or more steps of a function as shown in method 300. One or more portions of a method 300 may be performed by the processor executing any of the contents of memory, such as image processing 120, tracking 122, registration 124, and/or reconstruction 128.

The method 300 comprises tracking a catheter (step 304). The catheter may be the same as or similar to the catheter 136 and include a catheter tracker such as the catheter tracker 138. The catheter may be positioned or disposed in an anatomical element of a patient such as, for example, a heart such as the heart 206. In other embodiments, the catheter may be positioned in any portion or anatomical element of the patient. The catheter tracker may be used to track the catheter as the catheter moves whether due to a user moving the catheter and/or as a result of a cyclic heartbeat and/or respiratory movement of the patient. The catheter tracker may comprise one or more sensors to sense the movement and/or pose of the catheter. In some embodiments, the catheter tracker comprises EM trackers and/or IMU trackers. The catheter tracker 138 may be tracked by, for example, a navigation system such as the navigation system 118. In some embodiments, a processor such as the processor 104 or a processor of the navigation system may execute a tracking such as the tracking 122 to track the catheter tracker.

The method 300 also comprises receiving sensor data corresponding to a plurality of pose information of the catheter (step 308). The sensor data is received from the catheter tracker and may be stored in, for example, memory such as the memory 106 and/or a database such as the database 130. The sensor data may be received wirelessly from the catheter tracker by, for example, the navigation system. The sensor data can be received continuously in real-time or near real-time. The sensor data can also be received at a predetermined interval or based on user input. The sensor data may be used by, for example, the processor to obtain the plurality of pose information. The pose information may include information such as a position and orientation of the catheter, coordinates of the catheter, etc. The sensor data may also be used to obtain other information such as, for example, a velocity of the catheter.

The method 300 also comprises determining one or more phases of a motion of an anatomical element based on the plurality of pose information (step 312). The one or more phases of motion may be determined by, for example, the processor mapping or graphing the movement of the catheter and identifying repeated patterns of the movement. The processor may then correlate the patterns of the movement with the one or more phases of motion. For example, in embodiments where the anatomical element comprises the heart or an organ adjacent to or near the heart, the repeated patterns may correspond to one or more phases of the heartbeat or cardiac cycle. More specifically, the one or more phases may comprise at least a diastole motion and a systole motion, which can be identified and correlated to the patterns of movement. The processor may also use artificial intelligence or machine learning to identify the one or more phases. In such embodiments, a plurality of training poses may be provided to the processor, and each set of training poses may be annotated to include identifying information about the corresponding one or more phases.

It will be appreciated that in alternative or additional embodiments the one or more phases of motion may correspond to a respiratory motion caused by the patient breathing. In such embodiments, the respiratory motion may be determined from the plurality of pose information and/or from tracking a reference marker such as the reference marker 140, which may be external to the patient.

It is beneficial to determine the one or more phases (e.g., the diastole motion and/or the systole motion) as images can be taken during a target phase (or phases) that produce higher quality images than images taken during an undesired phase, as will be described in detail below.

The method 300 also comprises receiving an image at a target phase of the one or more phases (step 316). The target phase may be selected automatically by, for example, the processor of the computing device or the navigation system. In other instances, the target phase may be selected by a user such as a surgeon or other medical provider. The target phase may be selected to reduce artifacts in the image. For example, in embodiments where the anatomical element comprises a heart, the target phase may comprise the diastole phase as there is less movement associated with the diastole phase as compared to the systole phase. It may also be desirable to receive an image or multiple images at the target phase to update a 3D model or display the image(s) at the target phase for consistency.

The image may be received or obtained from an imaging device such as the imaging device 112 and may be stored in the memory of a computing device such as the computing device 102. An imaging device may be any imaging device such as an MRI scanner, a CT scanner, any other X-ray based imaging device, or an ultrasound imaging device. The image may also be generated by and/or uploaded to any other component of a system such as the system 100. In some embodiments, the image may be indirectly received via any other component of the system or a node of a network to which the system is connected.

The image may be a 2D image or a 3D image or a set of 2D and/or 3D images. The image may depict a patient's anatomy or portion thereof and may include at least a portion of the anatomical element and/or the catheter. It will be appreciated that in some embodiments, the image may also depict a reference marker such as the reference marker 140. In some embodiments, the image may depict multiple anatomical elements associated with the patient anatomy, including incidental anatomical elements (e.g., ribs or other anatomical objects on which a surgery or surgical procedure will not be performed) in addition to target anatomical elements (e.g., vertebrae or other anatomical objects on which a surgery or surgical procedure is to be performed). The image may comprise various features corresponding to the patient's anatomy and/or anatomical elements (and/or portions thereof), including gradients corresponding to boundaries and/or contours of the various depicted anatomical elements, varying levels of intensity corresponding to varying surface textures of the various depicted anatomical elements, combinations thereof, and/or the like. The image may depict any portion or part of patient anatomy and may include, but is in no way limited to, one or more vertebrae, ribs, lungs, soft tissues (e.g., skin, tendons, muscle fiber, etc.), a patella, a clavicle, a scapula, combinations thereof, and/or the like.

Each image may be processed by the processor using an image processing such as the image processing 120 to identify anatomical elements and/or the reference marker in the image. In some embodiments, feature recognition may be used to identify a feature of the anatomical element or the reference marker. For example, a contour of a vertebrae, femur, or other bone may be identified in the image. In other embodiments, the image processing algorithm may use artificial intelligence or machine learning to identify the anatomical element and/or the reference marker. In such embodiments, a plurality of training images may be provided to the processor, and each training image may be annotated to include identifying information about a reference marker and/or an anatomical element in the image.

The method 300 also comprises registering the image (step 320). The image may be registered by the processor using a registration such as the registration 124. The registration may transform, map, or create a correlation between the image received in the step 320 and/or components thereof, which may then be used by a system such as the system 100 or the navigation system to translate one or more coordinates in the patient coordinate space to one or more coordinates in a robot coordinate space of a robot (e.g., a robot 114) and/or vice versa. The registration may comprise registering between a 2D image and another 2D image and/or vice versa and/or between a 3D image (e.g., a CT scan) and one or more 2D images (e.g., ultrasound images) and/or vice versa.

In some embodiments, the registration may be performed one or more times intraoperatively (e.g., during surgery) to update, adjust, and/or refresh the current registration. For example, a new 3D image and/or a new plurality of 2D images may be captured intraoperatively, and a new registration may be completed therefrom (e.g., using a preoperative 3D image and a new plurality of intraoperative 2D images, a new intraoperative 3D image and a new plurality of 2D images, or otherwise). An updated registration may be required, for example, if a pose of the patient changes or is changed during the course of a surgical procedure. The registration may also be performed and/or repeated for a target phase or an updated target phase. For example, the registration may be completed for a first phase and later updated for a second phase.

The method 300 also comprises displaying the image (step 324). In some embodiments, after the step 320, the image may be displayed on a user interface such as the user interface 110 or a display such as the display 200. The image may be displayed at a target phase such as, for example, by strobing or freezing images (which may be, for example, ultrasound images) at the target phase, using a dynamic strobe effect to freeze image(s) at the target phase in the cardiac rhythm while allowing dynamic movement of implantable devices or instruments (e.g., the catheter, a cardiac device, etc.) relative to the anatomical element, hiding images taken outside of the target phase, highlighting image(s) of the target phase and diming image(s) outside of the target phase, and/or matching the target phase of a 3D image (e.g., CT or MRI) at a target phase. Such selective displaying of the image may visually aid a user such as a surgeon or other medical provide during placement of, for example, an implantable device such as a cardiac device, a stent, etc. More specifically, by displaying the image at a target phase, the image of the anatomical element may appear to be near stationary such that the user can maneuver the implantable device throughout the anatomical element more easily. Without the selective display at the target phase, the anatomical element would appear to be constantly moving due to the cyclic motion, which may make it difficult to visually maneuver the implantable device through the constantly moving anatomical element.

It will be appreciated that the steps 316, 320, and/or 324 may be repeated such that more than one image may be displayed throughout a surgical procedure. In some embodiments, images may be continuously received and displayed so as to provide real-time or near real-time imaging of the anatomical element, the catheter, and/or the implantable device. In other embodiments, image(s) may be updated at a predetermined time period. In still other embodiments, image(s) may be updated based on user input.

The method 300 also comprises reconstructing a 3D model (step 328). The 3D model may be reconstructed or updated based on the image received in the step 316. The 3D model may be reconstruction by the processor using a reconstruction such as the reconstruction 128. As previously described, generating the 3D representation by the processor 104 may include determining a surface representation or virtual boundary of the one or more anatomical elements and/or the one or more objects depicted in image(s) received in, for example, the step 316 and based on corresponding image pose information. The image pose information may be obtained from, for example, a robot such as the robot 114 supporting the imaging device. In some embodiments, each image may be positioned adjacent to another image based on the respective corresponding image pose information and a surface representation may be formed based the relative position of surfaces depicted in each image. In some embodiments, the surface representation may be a virtual mesh. The virtual mesh may comprise, for example, a set of polygonal faces that, when taken together, form a surface covering of a virtual object. The set of polygonal faces may be connected at their edges and vertices to define a shape of the virtual object.

Similarly to the step 324, the 3D model may be reconstructed using images from a target phase. For example, a first image from a first angle and a second image from a second angle may be taken at the same target phase such that portions of the anatomical element reconstructed from the first image and the second image are of similar position, size, and shape. In other words, if the first image and the second image are taken at different phases, the portions of the anatomical element depicted in the first image and the second image may be of different positions, sizes, and/or shapes and may not match up to each other. Thus, it is beneficial to obtain images from the same target phase(s) to reconstruct the 3D model. It will be appreciated that images from more than one phase can be used to reconstruct the 3D model in multiple phases.

The present disclosure encompasses embodiments of the method 300 that comprise more or fewer steps than those described above, and/or one or more steps that are different than the steps described above.

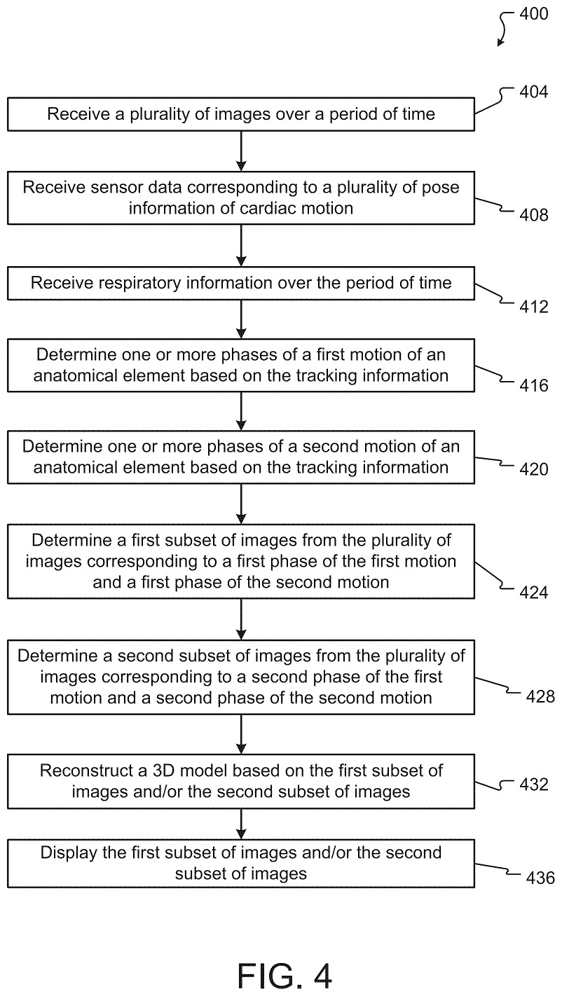

FIG. 4 depicts a method 400 that may be used, for example, for tracking movement of an anatomical element.

The method 400 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 400. The at least one processor may perform the method 400 by executing elements stored in a memory such as the memory 106. The elements stored in memory and executed by the processor may cause the processor to execute one or more steps of a function as shown in method 400. One or more portions of a method 400 may be performed by the processor executing any of the contents of memory, such as image processing 120, tracking 122, registration 124, and/or reconstruction 128.

The method 400 comprises receiving a plurality of images over a period of time (step 404). The step 404 may be the same as or similar to the step 316 of the method 300 described above, with the plurality of images obtained over the period of time.

The method 400 also comprises receiving sensor data corresponding to a plurality of pose information of cardiac motion over the period of time (step 408). In embodiments where the plurality of pose information is received from catheter trackers such as the catheter trackers 138 of a catheter such as the catheter 136, the step 408 may be the same as or similar to the step 308 of the method 300 described above except that the sensor data corresponding to the plurality of pose information is received over the period of time.

In other embodiments, the sensor data may comprise data from, for example, an EKG or ECG from which the one or more phases of the cardiac movement can be determined from. In some embodiments, the plurality of pose information may be determined from a combination of sensor data from the catheter trackers and/or from the EKG or ECG data. The sensor data and/or pose information may also be derived from image data (received from, for example, the imaging device) containing one or more catheters within the imaged field of view of the image data.

The method 400 also comprises receiving respiratory information over the period of time (step 412). The respiratory motion may be determined from the plurality of pose information received in the step 408 and/or from tracking a reference marker such as the reference marker 140 positioned on a patient. The reference marker may be external to the patient. In other embodiments, a sensor such as an EM and/or IMU sensor may be placed on or positioned in the patient anatomy to track, for example, movement of the thoracic or abdominal muscles which may correspond to respiratory movement.

The method 400 also comprises determining one or more phases of a first motion of an anatomical element based on the plurality of pose information (step 416). The step 416 is the same as or similar to the step 312 of the method 300 described above, except that the first motion may correspond to a heartbeat or cardiac cycle.

The method 400 also comprises determining one or more phases of a second motion of an anatomical element based on the respiratory information (step 420). The step 420 is the same as or similar to the step 312 of the method 300 described above, except that the second motion may correspond to a breathing or respiratory cycle. Further, the one or more phases may correspond to inhalation or exhalation during a respiratory cycle.

The method 400 also comprises determining a first subset of images from the plurality of images corresponding to a first phase of the first motion and a first phase of the second motion (step 424). The first subset of images may be determined by, for example, the processor determining each image that corresponds to the first phase of the first motion (e.g., a first phase of a cardiac cycle) and the first phase of the second motion (e.g., a first phase of a respiratory cycle). More specifically, the processor may correlate the plurality of images taken over the period of time with the plurality of pose information and the respiratory information over the period of time. Thus, each image can be correlated to a phase of the cardiac cycle and a phase of the respiratory cycle based on the corresponding pose information and respiratory information. Images of the same phase of the cardiac cycle and the respiratory cycle may then be grouped together. It will be appreciated that in some instances the cardiac cycle and the respiratory cycle may be synchronized. In other instances, the cardiac cycle and the respiratory cycle may occur in different frequencies and may not be synchronized. In other words, the images may correspond to different combinations of the cardiac cycle and the respiratory cycle at different subset of images.

The method 400 also comprises determining a second subset of images from the plurality of images corresponding to a second phase of the first motion and a second phase of the second motion (step 428). The step 428 may be the same as or similar to the step 424, except that the second subset of images is different from the first subset of images.

The method 400 also comprises reconstructing a 3D model based on the first subset of images and/or the second subset of images (step 432). The step 432 may be the same as or similar to the step 328 of the method 300 described above. It will be appreciated that in some embodiments, the 3D model may be reconstructed based on the first subset of images for the first phase of the first motion and the first phase of the second motion and the second subset of images for the second phase of the first motion and the second phase of the second motion. In some embodiments the first subset of images may correspond to images collected during a specific phase of the cardiac cycle and the second subset of images may correspond to images collected during a specific phase of the respiration cycle. For example, the first subset of images may correspond to a first subset of images collected in a diastole position during the cardiac cycle and the second subset of images may correspond to a second subset of images collected in an expiration position of the respiration cycle. The 3D model may then be reconstructed based on a combination of the first subset of images and the second subset of images.

The method 400 also comprises displaying the first subset of images and/or the second subset of images (step 436). The step 436 may be the same as or similar to the step 324 of the method 300 described above. Similarly to the step 436, the first subset of images for the first phase of the first motion and the first phase of the second motion may be displayed and the second subset of images for the second phase of the first motion and the second phase of the second motion may be separately displayed. In other words, multiple versions of the 3D model may be reconstructed for different phases of the first motion and/or the second motion.

It will be appreciated that any step and any combination of steps of the method 400 may be repeated. For example, the step 424 or 428 may be repeated for a third subset of images or any subsequent set(s) of images. Likewise, the step 432 and/or 436 may be repeated based on the third subset of images or any subsequent set(s) of images.

The present disclosure encompasses embodiments of the method 400 that comprise more or fewer steps than those described above, and/or one or more steps that are different than the steps described above.

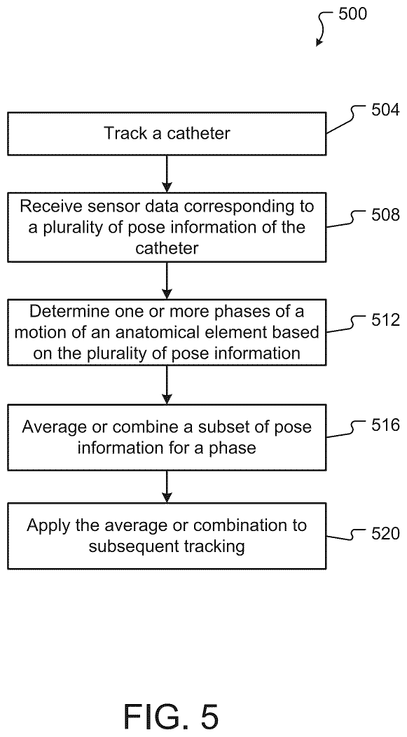

FIG. 5 depicts a method 500 that may be used, for example, for improving tracking of a catheter tracker.

The method 500 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 500. The at least one processor may perform the method 500 by executing elements stored in a memory such as the memory 106. The elements stored in the memory and executed by the processor may cause the processor to execute one or more steps of a function as shown in method 500. One or more portions of a method 500 may be performed by the processor executing any of the contents of memory, such as image processing 120, tracking 122, registration 124, and/or reconstruction 128.

The method 500 comprises tracking the catheter (step 504). The step 504 may be the same as or similar to the step 304 of the method 300 described above.

The method 500 also comprises receiving sensor data corresponding to a plurality of pose information of the catheter (step 508). The step 408 may be the same as or similar to the step 308 of the method 300 described above. Similarly, the sensor data and/or pose information may also be derived from image data (received from, for example, the imaging device) containing one or more catheters within the imaged field of view of the image data.

The method 500 also comprises determining one or more phases of a motion of an anatomical element based on the plurality of pose information (step 512). The step 512 may be the same as or similar to the step 312 of the method 300 described above.

The method 500 also comprises averaging or combining the sensor data for a target phase (step 516). The sensor data for the target phase may be averaged by, for example, a processor such as the processor 104. In other embodiments, the sensor data may be correlated or combined for a target phase or a plurality of target phases. By combining or averaging the sensor data for a target phase, variations or noise in the sensor data can be reduced, thereby increasing an accuracy of subsequent tracking for a target phase.

The method 500 also comprises applying the average or combination to subsequent tracking (step 520). By applying the average or combination of sensor data to the subsequent tracking, an accuracy of the tracking may be improved as the variations in individual sensor data can be reduced. It will be appreciated that the steps 516 and 520 may be repeated and act as a feedback loop to increase an accuracy of the tracking of the catheter device throughout, for example, a surgical procedure.

The present disclosure encompasses embodiments of the method 500 that comprise more or fewer steps than those described above, and/or one or more steps that are different than the steps described above.

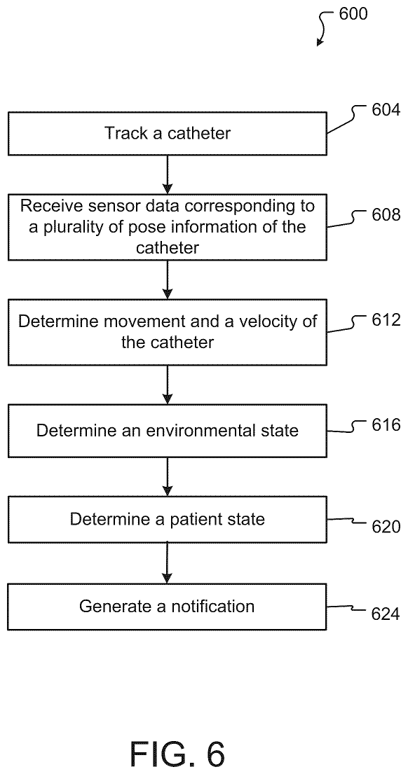

FIG. 6 depicts a method 600 that may be used, for example, for determining a patient state of environmental state of a catheter or a patient state.

The method 600 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 600. The at least one processor may perform the method 600 by executing elements stored in a memory such as the memory 106. The elements stored in the memory and executed by the processor may cause the processor to execute one or more steps of a function as shown in method 600. One or more portions of a method 600 may be performed by the processor executing any of the contents of memory, such as image processing 120, tracking 122, registration 124, and/or reconstruction 128.

The method 600 comprises tracking the catheter (step 604). The step 604 may be the same as or similar to the step 304 of the method 300 described above.

The method 600 also comprises receiving sensor data corresponding to a plurality of pose information of the catheter (step 608). The step 608 may be the same as or similar to the step 308 of the method 300 described above.

The method 600 also comprises determining a velocity of the catheter (step 612). The velocity of the catheter may be determined by a processor such as the processor 104 using the plurality of pose information of the catheter over a period of time. It will be appreciated that movement or velocity of the catheter may have different characteristics based on which portion of the anatomical element that the catheter is positioned in. For example, in embodiments where the anatomical element is the heart, the catheter may have more or less movement in a center of the aorta as compared to near a wall of the aorta. In another example, motion of the catheter in the ventricle should be different than motion in the atrium. Such information may be useful in confirming a position of the catheter within the anatomical element and thus, confirming placement of an implantable device such as a cardiac device in a desired position. The information may also be useful in determining whether the environmental state of the catheter is suitable for the implantable device and/or a patient state, as will be described below.

The method 600 also comprises determining an environmental state (step 616). The environmental state may be determined using, for example, the velocity of the catheter determined in the step 612. In some embodiments, the environmental state may also be determined using, for example, pressure that may be measured by, for example, the catheter tracker. The environmental state may correlate to an environmental state of the catheter positioned in the anatomical element. The environmental state may be useful in determining whether the location in which the catheter is positioned is suitable for, for example, a cardiac device or any other implantable device. For example, abnormal velocity of the catheter may indicate motion that may damage or interfere with an implantable device. On the other hand, velocity that correlates to an expected velocity indicates that the placement is sufficient for the implantable device. The velocity of the catheter may also be used to determine if motion of the catheter (and thus, the implantable device) may exceed force and/or torque limits on the implantable device. Thus, if the environmental state is insufficient, the implantable device may be implanted in another location or a different implantable device may be selected.

The method 600 also comprises determining a patient state (step 620). The patient state may be determined using, for example, the velocity of the catheter determined in the step 612. The patient state may correlate to a state of the patient and whether the patient may, for example, have any disease states. For example, low velocity or motion of the catheter may indicate low blood flow rates. In another example, pathologies may be determined based on movement of the catheter in a specific location.

In embodiments where two or more catheter trackers are positioned on the catheter, a differential between the two catheter trackers (e.g., a relative spacing and length of flow path between the two catheter trackers) can be used to determine a pulse wave velocity. The pulse wave velocity may correlate to a stiffness of a vessel wall of the anatomical element and more specifically, can be used to estimate a degree of arterial disease. For example, an increased pulse wave velocity may indicate plague in the vessel wall as the vessel wall will be thicker and thus, increase the pulse wave velocity.

The method 600 also comprises generating a notification (step 624). The notification may be a visual notification, an audible notification, or any type of notification communicated to a user. The notification may be communicated to the user via a user interface such as the user interface 110. In some embodiments, the notification may be automatically generated by a processor such as the processor 104. In other embodiments, the notification may be automatically generated by any component of a system such as the system 100.

The notification may be based on the velocity (as determined in, for example, the step 612) exceeding a predetermined threshold or being less than the predetermined threshold. The notification may be alternatively or additionally based on the environmental state and/or the patient state being of an undesired environmental state and/or patient state. For example, the notification may be generated when the environmental state is determined to be insufficient for the implantable device. In another example, the notification may be generated when the patient state indicates that the patient may have a disease. Thus, the notification may alert a surgeon or user of an expected velocity, environmental state, and/or patient state that the surgeon or other user may wish to avoid or otherwise mitigate.

The present disclosure encompasses embodiments of the method 600 that comprise more or fewer steps than those described above, and/or one or more steps that are different than the steps described above.

As noted above, the present disclosure encompasses methods with fewer than all of the steps identified in FIGS. 3, 4, 5, and 6 (and the corresponding description of the methods 300, 400, 500 and 600), as well as methods that include additional steps beyond those identified in FIGS. 3, 4, 5, and 6 (and the corresponding description of the methods 300, 400, 500 and 600). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for determining one or more phases of anatomical movement comprising:
a catheter positioned in an anatomical element;
a tracker for tracking a pose of the catheter;
a processor; and
a memory storing data for processing by the processor, the data, when processed, causing the processor to:
track the catheter;
receive sensor data corresponding to a plurality of pose information of the catheter from the tracker;
determine one or more phases of motion of the anatomical element based on the plurality of pose information;
receive an image at a target phase of the one or more phases; and
display the image.

2. The system of claim 1, wherein tracking the catheter comprises tracking a speed of the catheter.

3. The system of claim 1, wherein at least one of the tracker, the catheter, or a combination of the tracker and the catheter is shaped to at least one of augment, decrease, or increase motion caused by fluid flow in the anatomical element.

4. The system of claim 1, wherein the memory stores further data for processing by the processor that, when processed, causes the processor to:
receive bio-electric signals, wherein determining the one or more phases of motion is further based on the bio-electric signals.

5. The system of claim 1, wherein the memory stores further data for processing by the processor that, when processed, causes the processor to:
receive respiratory cycle signals, wherein determining the one or more phases of motion is further based on the bio-electric signals.

6. The system of claim 1, wherein the anatomical element comprises a heart and the one or more phases correspond to one or more phases of the heartbeat.

7. The system of claim 6, wherein the one or more phases of the heartbeat comprise at least a diastole motion and a systole motion.

8. The system of claim 1, wherein the memory stores further data for processing by the processor that, when processed, causes the processor to:
average the sensor data for a target phase of the one or more phases; and
apply the average to subsequent tracking at the target phase.

9. The system of claim 1, wherein the memory stores further data for processing by the processor that, when processed, causes the processor to determine a patient state based on the motion of the anatomical element.

10. The system of claim 1, wherein the memory stores further data for processing by the processor that, when processed, causes the processor to:
determine movement and a velocity of the catheter; and
determine an environmental state of the catheter based on the movement and velocity.

11. The system of claim 1, wherein the tracker comprises at least one of an inertial measurement unit tracker, an electromagnetic tracker, or a combination of the inertial measurement unit tracker and the electromagnetic tracker.

12. The system of claim 1, wherein the tracker comprises an imaging device and the sensor data comprises image data, wherein the plurality of pose information is derived from the image data.

13. A system for determining one or more phases of anatomical movement comprising:
a catheter positioned in an anatomical element;
a tracker for tracking a pose of the catheter;
a processor; and
a memory storing data for processing by the processor, the data, when processed, causing the processor to:
track the catheter;
receive sensor data corresponding to a plurality of pose information of the catheter from the tracker; and
determine one or more phases of motion of the anatomical element based on the plurality of pose information.

14. The system of claim 13, wherein the memory stores further data for processing by the processor that, when processed, causes the processor to receive an image at a phase of the one or more phases and display the image.

15. The system of claim 13, wherein the tracker comprises at least one of an inertial measurement unit tracker or an electromagnetic tracker.

16. The system of claim 13, wherein the memory stores further data for processing by the processor that, when processed, causes the processor to determine a patient state based on the motion of the anatomical element.

17. The system of claim 13, wherein the memory stores further data for processing by the processor that, when processed, causes the processor to:
determine movement and a velocity of the catheter; and
determine an environmental state of the catheter based on the movement and velocity.

18. A system for correlating one or more images with one or more cycles of motion of an anatomical element comprising:
a processor; and
a memory storing data for processing by the processor, the data, when processed, causing the processor to:
receive a plurality of images over a period of time;
receive sensor data corresponding to a plurality of pose information pose of a catheter corresponding to pose information of the catheter over the period of time to yield;
determine one or more first motion phases of a first motion of the anatomical element based on the plurality of pose information;
determine one or more second motion phases of a second motion of the anatomical element based on the plurality of pose information; and
determine a subset of images from the plurality of images corresponding to a target first motion phase of the one or more first motion phases of the first motion and a target second motion phase of the one or more second motion phases of the second motion.

19. The system of claim 18, wherein the subset of images comprises a first subset of images, and wherein the memory stores further data for processing by the processor that, when processed, causes the processor to determine a second subset of images from the plurality of images corresponding to another target first motion phase the one or more first motion phases of the first motion and another target second motion phase of the one or more second motion phases of the second motion.

20. The system of 19, wherein the memory stores further data for processing by the processor that, when processed, causes the processor to reconstruct a three-dimensional model based on an intersection of the first subset of images and the second subset of images.

21. The system of 18, wherein the memory stores further data for processing by the processor that, when processed, causes the processor to display at least one of the first subset of images, the second subset of images, or a combination of the first subset of images and the second subset of images.

22. The system of claim 18, wherein the first motion corresponds to a cardiac cycle and the second motion corresponding to a respiratory cycle.

23. The system of claim 18, wherein the memory stores further data for processing by the processor that, when processed, causes the processor to receive respiratory information of a patient over the period of time, wherein determining the one or more second motion phases of a second motion is also based on the respiratory information.

24. The system of claim 18, wherein the memory stores further data for processing by the processor that, when processed, causes the processor to obtain and display a subsequent plurality of images over a subsequent period of time at the target first motion phase and the target second motion phase.

25. The system of claim 18, wherein the memory stores further data for processing by the processor that, when processed, causes the processor to:
average sensor data at a target phase of the one or more phases; and
apply the average to subsequent tracking at the target phase.

26. The system of claim 18, wherein the sensor data comprises at least one of image data, accelerometer data, pose data, or motion data.

27. The system of claim 18, wherein the memory stores further data for processing by the processor that, when processed, causes the processor to:
receive bio-electric signals, wherein determining the one or more phases of motion is further based on the bio-electric signals.

28. The system of claim 18, wherein the memory stores further data for processing by the processor that, when processed, causes the processor to:
receive respiratory cycle signals, wherein determining the one or more phases of motion is further based on the bio-electric signals.
